(19) United States
(12) Patent Application Publication
(10) Pub. No.: US 2013/0123545 A1
(43) Pub. Date: May 16, 2013

(54) METHOD FOR SYNTHESIZING DIKETOPYRACENE

(71) Applicants: Kei Tagami, Yokohama-shi (JP); Masashi Hashimoto, Tokyo (JP); Hiroki Ohrui, Kawasaki-shi (JP)

(72) Inventors: Kei Tagami, Yokohama-shi (JP); Masashi Hashimoto, Tokyo (JP); Hiroki Ohrui, Kawasaki-shi (JP)

(73) Assignee: CANON KABUSHIKI KAISHA, Tokyo (JP)

(21) Appl. No.: 13/812,475

(22) PCT Filed: Jul. 20, 2011
(86) PCT No.: PCT/JP2011/067019

§ 371 Date: Jan. 25, 2013

(30) Foreign Application Priority Data
Jul. 30, 2010 (JP) 2010-172934

(51) Int. Cl. C07C 45/30

(52) U.S. Cl. CPC C07C 45/30
USPC 568/323

(57) ABSTRACT

Diketopyracene is synthesized in high yield without using carbon disulfide as a solvent. A special solvent is used. In particular, a solvent having a benzene ring that has two or more chlorine atoms is used. The two chlorine atoms are not in meta positions but in ortho positions of the benzene ring.

### TECHNICAL FIELD

The present invention relates to a method for synthesizing diketopyracene without using carbon disulfide as a solvent.

### BACKGROUND ART

Diketopyracene is represented by a structural formula below. Diketopyracene may hereinafter referred to as “compound 1”.

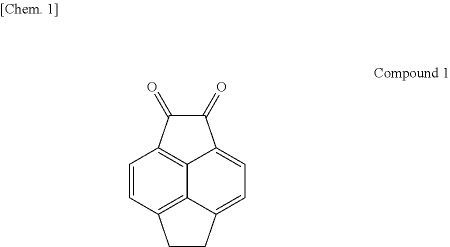 [SMILES: O=C1C(=O)c2ccc3c4c(ccc1c24)CC3]

In synthesizing diketopyracene, carbon disulfide is used as a solvent (NPL 1). In order to obtain diketopyracene, aluminum chloride or aluminum bromide is used as a catalyst. In NPL 1, carbon disulfide that can dissolve these catalysts is used as a solvent.

Since the flash point of carbon disulfide is −30° C., solvents other than carbon disulfide should be used in the industrial production of diketopyracene.

Moreover, according to the synthetic method disclosed in NPL 1, stirring stops during the reaction and the yield is as low as 17%.

### CITATION LIST

### Non Patent Literature

NPL 1 Journal of the American Chemical Society 91, 918 (1969)

### SUMMARY OF INVENTION

It is desirable to provide a method for synthesizing diketopyracene from acenaphthene in the presence of aluminum bromide, oxalyl bromide, and a solvent. The solvent is a liquid that dissolves aluminum bromide, oxalyl bromide, and acenaphthene and is represented by general formula (1):

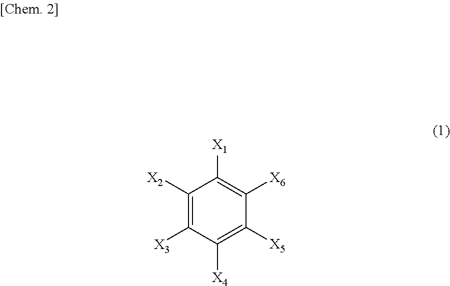

In general formula (1), X₁ to X₆ each independently denote a hydrogen atom, a chlorine atom, a fluorine atom, or a substituted or unsubstituted alkyl group and at least two adjacent groups selected from X₁ to X₆ each independently denote a chlorine atom or a fluorine atom.

The method includes one of A and B:

A: adding dropwise a solution containing acenaphthene, oxalyl bromide, and the solvent to a container containing a solution containing aluminum bromide and the solvent so as to mix the solutions,

B: adding dropwise a solution containing aluminum bromide and the solvent and a solution containing acenaphthene, oxalyl bromide, and the solvent simultaneously into a container so as to mix the solutions.

### DESCRIPTION OF EMBODIMENTS

A method for synthesizing diketopyracene according to an embodiment of the present invention is a method for obtaining diketopyracene from acenaphthene in the presence of a solvent, aluminum bromide, and oxalyl bromide. Diketopyracene is obtained by a reaction between acenaphthene and oxalyl bromide catalyzed by aluminum bromide.

Acenaphthene is represented by the following structure.

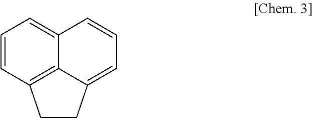 [SMILES: c1cc2c3c(cccc3c1)CC2]

Oxalyl bromide is represented by the following structure.

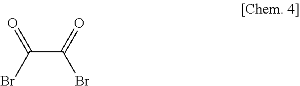

A compound X represented by general formula (1) is used as a solvent that dissolves aluminum bromide.

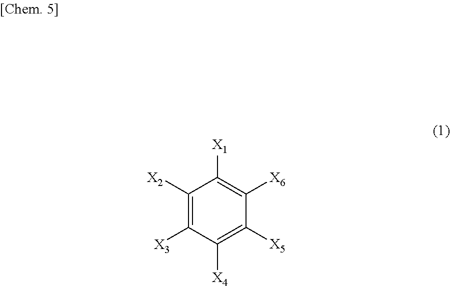

The reaction formula is described below. In this reaction, acylation occurs twice.

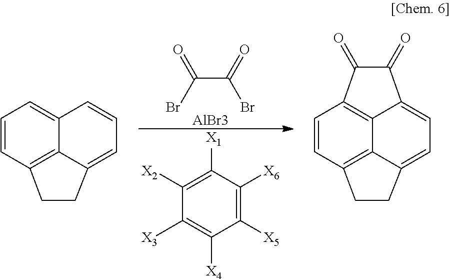

The order in which these three compounds (aluminum bromide, acenaphthene, and oxalyl bromide) are fed to a reaction field greatly affects the diketopyracene yield.

Before describing this order, the compound X used as a solvent is described.

The compound X is represented by general formula (1) below.

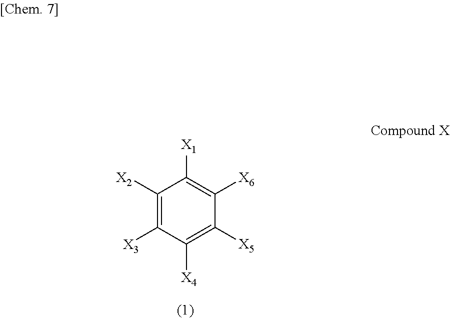

In general formula (1), X₁ to X₆ each independently denote a hydrogen atom, a chlorine atom, a fluorine atom, or a substituted or unsubstituted alkyl group. At least two adjacent groups selected from X₁ to X₆ each independently denote a chlorine atom or a fluorine atom.

When one of X₁ to X₆ is a substituted or unsubstituted alkyl group, the substituted or unsubstituted alkyl group may be any. Examples thereof include a methyl group, an ethyl group, a propyl group, an isopropyl group, a butyl group, a sec-butyl group, a tert-butyl group, a pentyl group, a hexyl group, and an octyl group. With these alkyl groups, the diketopyracene yield may be in the range of 15% to 45%.

The compound X used for synthesis is not limited to one type. A mixture of two or more compounds may be used as the compound X.

In the compound X, at least two adjacent groups selected from X₁ to X₆ each independently denote a chlorine atom or a fluorine atom.

In contrast, when a compound other than the compound X is used, i.e., when a compound having a skeleton represented by general formula (1) in which the combination of at least two adjacent atoms in X₁ to X₆ is other than chlorine/chlorine, chlorine/fluorine, and fluorine/fluorine is used, the diketopyracene yield drops significantly. This applies even when chlorine and fluorine atoms are at meta or para positions.

The compound X in the present invention may include three or more chlorine atoms and/or fluorine atoms.

Specific examples of the compound X represented by general formula (1) and used in the present invention include, but are not limited to, the following: orthodichlorobenzene, 1,2-difluorobenzene, 2-chlorofluorobenzene, 1,2,3-trichlorobenzene, 1,2,4-trichlorobenzene, 1,2,3-trifluorobenzene, 1,2,4-trifluorobenzene; 1,2-dichloro-4-fluorobenzene, 1,2-dichloro-3-fluorobenzene, 1,3-dichloro-2-fluorobenzene, 1,4-dichloro-2-fluorobenzene, 2,4-dichloro-1-fluorobenzene; 1-chloro-2,3-difluorobenzene, 1-chloro-2,4-difluorobenzene, 2-chloro-1,3-difluorobenzene, 2-chloro-1,4-difluorobenzene, 4-chloro-1,2-difluorobenzene; 1,2-dichloro-4-methylbenzene, 1,2-dichloro-4-ethylbenzene, 1,2-dichloro-4-propylbenzene, 1,2-dichloro-4-isopropylbenzene, 4-tert-butyl-1,2-dichlorobenzene; 2-chloro-1-fluoro-4-methylbenzene, 2-chloro-1-fluoro-4-ethylbenzene, 2-chloro-1-fluoro-4-propylbenzene, 2-chloro-1-fluoro-4-iropropylbenzene, 4-tert-butyl-2-chloro-1-fluorobenzene; 1-chloro-2-fluoro-4-methylbenzene, 1-chloro-2-fluoro-4-ethylbenzene, 1-chloro-2-fluoro-4-propylbenzene, 1-chloro-2-fluoro-4-isopropylbenzene, 4-tert-butyl-1-chloro-2-fluorobenzene; 1,2-difluoro-4-methylbenzene, 1,2-difluoro-4-ethylbenzene, 1,2-difluoro-4-propylbenzene, 1,2-difluoro-4-isopropylbenzene, 4-tert-butyl-1,2-difluorobenzene; and 1,2,3,4-tetrachlorobenzene, 1,2,3,5-tetrachlorobenzene, 1,2,3,4-tetrafluorobenzene, 1,2,3,5-tetrafluorobenzene, and 1,2,4,5-tetrafluorobenzene.

The order of feeding the compounds is described next.

The order described in NPL 1 is that oxalyl bromide is first added to the solution of acenaphthene in a solvent and then aluminum bromide. According to this order, the yield is low.

The inventor has found that diketopyracene can be obtained in high yield when one of process A and process B below is conducted.

### Process A:

The inventor has found that it is desirable to add dropwise a solution of acenaphthene and oxalyl bromide in the compound X to a solution of aluminum bromide in the compound X in a container.

### Process B:

The inventor has also found that the compound 1 can be obtained in high yield by adding dropwise a solution of aluminum bromide in the compound X and a solution of acenaphthene and oxalyl bromide in the compound X to a container (e.g., the two solutions may be added simultaneously).

The common feature of the process A and the process B is that the equivalent weight of aluminum bromide is larger than the equivalent weight of acenaphthene and the equivalent weight of oxalyl bromide in the reaction field (reaction solution) from the beginning (from the start of mixing). This improves the yield.

In particular, in the process B, aluminum bromide may be added dropwise in excess relative to acenaphthene and oxalyl bromide. Moreover, the lengths of time taken for the dropwise addition of the two solutions may be the same.

In the processes A and B, the length of time taken for dropwise addition is 45 minutes or longer to obtain diketopyracene in high yield.

In Examples, the process A is indicated as a feed method 1 and the process B is indicated as a feed method 2. The order described in NPL 1 is indicated as a feed method 3 hereinafter. The feed method 3 is employed in Comparative Examples.

The reaction conditions common to the processes A and B are as follows.

The reaction temperature may be in the range of 0° C. to 35° C. At less than 0° C., a longer reaction time is needed. At higher than 35° C., the amount of impurities generated tends to be large.

The amount of the compound X used is preferably 1% to 100% by weight and more preferably 8% to 20% by weight relative to acenaphthene.

The amount of aluminum bromide used is preferably larger the equivalent amounts of acenaphthene and oxalyl bromide. The amount of aluminum bromide is more preferably 2.0 equivalents or more per equivalent of acenaphthene used. When an excessive amount of aluminum bromide is used, the post-process operation becomes complicated. The amount of aluminum bromide used is more preferably 2.5 to 4.0 equivalents.

The amount of oxalyl bromide used is preferably smaller than the equivalent amount of aluminum bromide. The amount of oxalyl bromide is more preferably 1.0 to 2.0 equivalents relative to acenaphthene.

The reaction may be conducted under normal pressure. The reaction may be conducted in an inert atmosphere such as nitrogen or argon.

### EXAMPLES

Examples and Comparative Examples in which three feed methods were conducted with various different compounds X as a solvent are described below. These examples involve methods for synthesizing diketopyracene which is a compound 1. The three feed methods are the feed method 1, the feed method 2, and the feed method 3 discussed above.

Before describing Examples and Comparative Examples, determination of purity of diketopyracene is described. An absolute purity is a ratio of the content of a compound relative to the total amount of a solid. The absolute purity is measured and then a yield is determined from the total amount of the solid and the determined absolute purity.

The absolute purity was measured by subjecting a specimen to liquid chromatography and drawing a calibration curve. The amount of the compound 1 contained in the solid was calculated from the calibration curve and the results of sample measurement and then the yield is determined. The specimen was diketopyracene prepared by the method described below. Diketopyracene synthesized by the synthetic method of the invention is purified by silica gel chromatography (developing solvent: chloroform) and recrystallized with chloroform to obtain the specimen. A “sample” refers to a substance obtained in each of Examples and Comparative Examples. The phrase “diketopyracene is contained in the solid” means that diketopyracene is contained in an organic or inorganic material.

Liquid chromatography used in measuring the absolute purity is used to draw a calibration curve and is used in measuring the purity of diketopyracene obtained through synthesis and purification.

The analysis by liquid chromatography used a column, ODS-H80 produced by YMC Co., Ltd., and an acetonitrile/water (8:2) mixed solvent as a solvent. Development was conducted at a solution flow rate of 1.0 mL/min and detection was conducted with 254 nm ultraviolet (UV) light. The desired purity can be found by detection. When a desired purity was obtained, the diketopyracene content was determined from the calibration curve of the specimen and the ratio of the diketopyracene content relative to the total amount of the solid was calculated to determine the absolute purity.

Table 1 shows the solvents used, the diketopyracene yield, and the method selected in Examples and Comparative Examples.

TABLE 1

| | | | Feed |
|---|---|---|---|
| | | | meth- |
| | Solvent (compound X) | Yield | od |
| Example 1 | 1,2,4-Trichlorobenzene | 42.4% | 1 |
| Example 2 | 1,2,4-Trichlorobenzene | 40.1% | 2 |
| Example 3 | 1,2,4- | 33.1% | 1 |
| | Trichlorobenzene:orthodichlorobenzene = | | |
| | 1:1 | | |
| Example 4 | Orthodichlorobenzene | 20.8% | 1 |
| Comparative | Metadichlorobenzene | 7.90% | 1 |
| Example 1 | | | |
| Comparative | 1,2,4-Trichlorobenzene | 2.60% | 3 |
| Example 2 | | | |
| Comparative | Metadibromobenzene | — | 1 |
| Example 3 | | | |
| Comparative | Chloroform | 1.80% | 3 |
| Example 4 | | | |
| Comparative | Dichloromethane | — | 3 |
| Example 5 | | | |
| Comparative | Nitrobenzene | — | 3 |
| Example 6 | | | |

Examples and Comparative Examples will now be described.

### Example 1

In Example 1, 1,2,4-trichlorobenzene was used as a solvent and diketopyracene was synthesized by the feed method 1.

Under nitrogen stream, 1.03 kg (3.86 mol) of aluminum bromide and 2.14 L of 1,2,4-trichlorobenzene were added to a reactor and stirred to dissolve aluminum bromide. To the reactor, a solution prepared by dissolving 238 g (1.54 mol) of acenaphthene and 349 g (1.62 mol) of oxalyl bromide in 0.714 L of 1,2,4-trichlorobenzene was added dropwise in 4 hours, followed by stirring for 1 hour. Upon completion of the stirring, 1.43 L of orthodichlorobenzene was added, the reaction solution was cooled to 0° C., and the reaction was terminated with a 10% aqueous sulfuric acid solution. After termination of the reaction, 6.42 L of isopropyl ether was added, followed by stirring for 1 hour and filtration. The residue was washed by being dispersed in water, isopropyl ether, and then toluene. As a result, 147 g of a brown solid, i.e., compound 1, was obtained. The absolute purity of the solid compound 1 was measured. The yield was 42.4%.

The structure was identified by nuclear magnetic resonance (NMR) spectroscopy. The distribution of the peaks was as follows:

¹H-NMR (300 MHz, CDCl₃): δ (ppm)=8.01 (d, 2H, J=6.0 Hz), 7.59 (d, 2H, J=6.0 Hz), 3.65 (s, 4H)

### Example 2

In Example 2, diketopyracene was synthesized by the feed method 2 using 1,2,4-trichlorobenzene as a solvent.

Under nitrogen stream, 3 mL of 1,2,4-trichlorobenzene was added to a reactor. To the reactor, a solution of 4.37 g (16.4 mmol) of aluminum bromide in 14.0 mL of 1,2,4-trichlorobenzene and a solution of 1.01 g (6.56 mmol) of acenaphthene and 1.48 g (6.87 mmol) of oxalyl bromide in 5.0 mL of 1,2,4-trichlorobenzene were added dropwise in 45 minutes, followed by stirring for 1 hour. Upon completion of stirring, 6.1 mL of orthodichlorobenzene was added, and the resulting mixture was cooled to 0° C. The reaction was terminated with a 10% aqueous sulfuric acid solution. After termination of the reaction, 27.3 mL of isopropyl ether was added, followed by stirring for 1 hour and filtration. The residue was washed by being dispersed in water, isopropyl ether, and then toluene. As a result, 0.61 g of a brown solid, compound 1, was obtained. The absolute purity of the solid compound 1 obtained was measured. The yield was 40.1%.

### Example 3

In Example 3, diketopyracene was synthesized by the feed method 1 using 1,2,4-trichlorobenzene and orthodichlorobenzene as solvents.

Under nitrogen stream, 4.76 g (17.8 mmol) of aluminum bromide, 5.0 mL of 1,2,4-trichlorobenzene, and 5.0 mL of orthodichlorobenzene were added to a reactor and stirred to dissolve aluminum bromide. Thereto, a solution of 1.10 g (7.13 mmol) of acenaphthene and 1.62 g (7.48 mmol) of oxalyl bromide in 1.6 mL of 1,2,4-trichlorobenzene and 1.6 mL of orthodichlorobenzene was added dropwise in 45 minutes, followed by stirring for 5 hours. Upon completion of stirring, the mixture was cooled to 0° C. and the reaction was terminated with a 10% aqueous sulfuric acid solution. After termination of the reaction, 29.8 mL of isopropyl ether was added, followed by stirring for 1 hour and filtration. The residue was washed by being dispersed in water, isopropyl ether, and then toluene. As a result, 0.55 g of a brown solid, compound 1, was obtained. The absolute purity of the solid compound 1 obtained was measured. The yield was 33.1%.

### Example 4

In Example 4, diketopyracene was synthesized by the feed method 1 using orthodichlorobenzene as a solvent.

Under nitrogen stream, 4.32 g (16.2 mmol) of aluminum bromide and 14.0 mL of orthodichlorobenzene were added to a reactor and stirred to dissolve aluminum bromide.

To the reactor, a solution of 1.00 g (6.40 mmol) of acenaphthene and 1.47 g (6.80 mmol) of oxalyl bromide in 6.0 mL of orthodichlorobenzene was added dropwise in 45 minutes, followed by stirring for 2 hours. Upon completion of stirring, the mixture was cooled to 0° C., and the reaction was terminated with a 10% aqueous sulfuric acid solution. After termination of the reaction, 26.9 mL of isopropyl ether was added, followed by stirring for 1 hour and filtration. The residue was washed by being dispersed in water, isopropyl ether, and then toluene. As a result, 0.31 g of a brown solid, compound 1, was obtained. The absolute purity of the solid compound 1 obtained was measured. The yield was 20.8%.

### Comparative Example 1

In Comparative Example 1, diketopyracene was synthesized by the feed method 1 using a solvent different from the compound X.

Under nitrogen stream, 5.27 g (19.7 mmol) of aluminum bromide and 14.0 mL of metadichlorobenzene were added to a reactor and stirred to dissolve aluminum bromide. To the reactor, a solution of 1.22 g (7.90 mmol) of acenaphthene and 1.79 g (8.30 mmol) of oxalyl bromide in 6.0 mL of metadichlorobenzene was added dropwise in 45 minutes, followed by stirring for 2 hours. Upon completion of the stirring, the mixture was cooled to 0° C. and the reaction was terminated with a 10% aqueous sulfuric acid solution. After the termination of the reaction, 32.9 mL of isopropyl ether was added, followed by stirring for 1 hour and filtration. The residue was washed by being dispersed in water, isopropyl ether, and then toluene. As a result, 0.14 g of a brown solid, compound 1, was obtained. The absolute purity of the solid compound 1 obtained was measured. The yield was 7.9%.

### Comparative Example 2

In Comparative Example 2, diketopyracene was synthesized by the feed method 3 using a solvent different from the compound X.

Under nitrogen stream, 1.04 g (6.72 mmol) of acenaphthene was fed into a reactor, and 12.5 mL of 1,2,4-trichlorobenzene was added thereto. After adding 1.53 g (6.72 mmol) oxalyl bromide in one batch, 4.50 g (16.8 mmol) of aluminum bromide was gradually added thereto in 30 minutes, followed by stirring for 1 hour. Upon completion of stirring, 6.2 mL of orthodichlorobenzene was added and the mixture was cooled to 0° C. The reaction was terminated with a 10% aqueous sulfuric acid solution. After the termination of the reaction, 28.0 mL of isopropyl ether was added, followed by stirring for 1 hour and filtration. The residue was washed by being dispersed in water, isopropyl ether, and then toluene. As a result, 0.092 g of a brown solid, compound 1, was obtained. The absolute purity of the solid compound 1 obtained was measured. The yield was 2.6%.

### Comparative Example 3

In Comparative Example 3, an attempt to synthesize diketopyracene was made by using the feed method 1 and a solvent different from the compound X.

Under nitrogen stream, 4.45 g (16.7 mmol) of aluminum bromide and 14.0 mL of metadibromobenzene were added to a reactor and stirred to dissolve aluminum bromide. A solution of 1.03 g (6.65 mmol) of acenaphthene and 1.51 g (7.01 mmol) of oxalyl bromide in 6.0 mL of metadibromobenzene was added to the reactor dropwise in 45 minutes, followed by stirring. The temperature increased during the stirring, resulting in solidification of the solution, and the stirring stopped.

### Comparative Example 4

In Comparative Example 4, diketopyracene was synthesized by the feed method 3 using a solvent different from the compound X.

Into a 1 L flask, 6.80 g (44.1 mmol) was fed under argon stream, and dehydrated chloroform (600 mL) was added thereto. After the mixture was cooled to 0° C. in ice water, 10.0 g (46.3 mmol) of oxalyl bromide was added in one batch, and 29.4 g (110 mmol) of aluminum bromide was gradually added thereto in 30 minutes. After the addition, the resulting mixture was stirred at the same temperature for 5 hours, and left standing still overnight. Then the reaction product was separated into a supernatant and wall deposits. The supernatant was analyzed by NMR and was found not to be the compound 1. The absolute purity of the compound 1 in the wall deposits was measured. The yield was 1.8%.

### Comparative Example 5

In Comparative Example 5, an attempt to synthesize diketopyracene was made by using the feed method 3 and a solvent different from the compound X.

Into a 1 L flask, 6.80 g (44.1 mmol) of acenaphthene was fed under argon stream, and dichloromethane (600 mL) was added thereto. After the mixture was cooled to 0° C. in ice water, 10.0 g (46.3 mmol) of oxalyl bromide was added in one batch, and 29.4 g (110 mmol) of aluminum bromide was gradually added in 30 minutes. After the addition, the resulting mixture was stirred at the same temperature for 5 hours and left standing still overnight. The reaction solution was analyzed by NMR and found not to be the compound 1.

### Comparative Example 6

In Comparative Example 6, an attempt to synthesize diketopyracene was made by using the feed method 3 and a solvent different from the compound X.

Into a 1 L flask, 6.80 g (44.1 mmol) of acenaphthene was fed under argon stream, and nitrobenzene (600 mL) was added thereto. After the mixture was cooled to 0° C. in ice water, 10.0 g (46.3 mmol) of oxalyl bromide was added in one batch, and 29.4 g (110 mmol) of aluminum bromide was gradually added thereto in 30 minutes. After the addition, the resulting mixture was stirred at the same temperature for 5 hours and left standing still overnight. The reaction solution was analyzed by NMR and found not to be the compound 1.

### Results

The yield was as high as 20% or more in all of Examples 1 to 4. In contrast, the yield was less than 10% in Comparative Examples 1, 2, and 4, and synthesis was failed in Comparative Examples 3, 5, and 6.

According to Examples, the diketopyracene yield is highest with 1,2,4-trichlorobenzene, second highest with orthodichlorobenzene, and third highest with metadichlorobenzene. This difference is presumably derived from the substitution positions and number of substitution sites for chlorine atoms. Comparison between Examples and Comparative Example 3 shows that the cause of difference in diketopyracene yield is presumably the difference between chlorine atoms and bromine atoms.

The difference is presumably derived from the difference in leaving ability between chlorine and bromine. As form carbon-halogen bonding, fluorine atoms are stronger than chlorine atoms and fluorine atoms presumably have the same effects as chlorine atoms. In other words, candidates for the halogen atom in the compound X may include a fluorine atoms in addition to the chlorine atoms described in Examples.

Diketopyracene may be used as a raw material for synthesizing a fluorescent compound. A fluorescent compound may be used as a compound for an organic electroluminescent (EL) device.

In sum, the embodiments and examples described above indicate that diketopyracene can be synthesized in high yield without using carbon disulfide.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2010-172934, filed Jul. 30, 2010, which is hereby incorporated by reference herein in its entirety.

1. A method for synthesizing diketopyracene from acenaphthene in the presence of aluminum bromide, oxalyl bromide, and a solvent, the solvent being a liquid that dissolves aluminum bromide, oxalyl bromide, and acenaphthene and being represented by general formula (1) 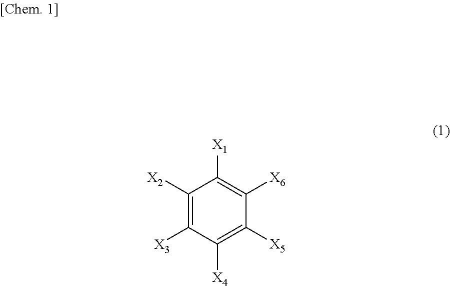
where X₁ to X₆ each independently denote a hydrogen atom, a chlorine atom, a fluorine atom, or a substituted or unsubstituted alkyl group and at least two adjacent groups selected from X₁ to X₆ each independently denote a chlorine atom or a fluorine atom, the method comprising one of A and B:
A: adding dropwise a solution containing acenaphthene, oxalyl bromide, and the solvent to a container containing a solution containing aluminum bromide and the solvent so as to mix the solutions,
B: adding dropwise a solution containing aluminum bromide and the solvent and a solution containing acenaphthene, oxalyl bromide, and the solvent simultaneously into a container so as to mix the solutions.

2. The method according to claim 1, wherein the solvent is one of 1,2,4-trichlorobenzene and orthodichlorobenzene.

3. The method according to claim 1, wherein aluminum bromide is used in an amount larger than the equivalent of acenaphthene and the equivalent of oxalyl bromide.

4. The method according to claim 1, wherein the amount of aluminum bromide used is 2.5 to 4.0 equivalents per equivalent of acenaphthene used and the amount of oxalyl bromide used is 1.0 to 2.0 equivalents per equivalent of acenaphthene.
